(19) United States
(12) Patent Application Publication
(10) Pub. No.: US 2025/0340503 A1
(43) Pub. Date: Nov. 6, 2025

(54) PHOTOINDUCED OXIDATION OF METHANE TO OXYGENATES

(71) Applicant: THE ROYAL INSTITUTION FOR THE ADVANCEMENT OF LEARNING/MCGILL UNIVERSITY, Montréal (CA)

(72) Inventors: Chao-Jun LI, Brossard (CA); Jingtan HAN, Montréal (CA); Hui SU, Montréal (CA)

(21) Appl. No.: 18/998,618

(22) PCT Filed: Jul. 26, 2023
(86) PCT No.: PCT/CA2023/051007

§ 371 Date: Jan. 27, 2025

Related U.S. Application Data
(60) Provisional application No. 63/392,619, filed on Jul. 27, 2022.

(51) Int. Cl. C07C 51/215; C07C 29/50

(52) U.S. Cl. CPC C07C 51/215; C07C 29/50

(57) ABSTRACT

There is provided a method of producing Cl oxygenated products with a GaN catalyst. The Cl oxygenated products are preferably methanol and formic acid. The method is optionally selective for methanol or formic acid. The GaN catalyst and an aqueous phase are provided in a reactor having a closed environment. 0.1 to 0.55 bar of methane and from 0 to 10 bar of oxygen are introduced in the reactor. The reactor is irradiated with ultra-violet light until Cl oxygenated products such as methanol and formic acid are obtained.

### CROSS-REFERENCE TO A RELATED APPLICATION

This disclosure claims the priority of U.S. provisional application No. 63/392,619 filed Jul. 27, 2022 and hereby incorporated by reference in its entirety.

### TECHNICAL FIELD

This disclosure relates to the field of methane oxidation to obtain oxygenates such as methanol and formic acid, and more specifically to a photo-catalyzed oxidation of methane to oxygenates.

### BACKGROUND OF THE ART

Methane (CH₄) can be found in natural gas, biogas, shale gas and ocean floors. Methane has drawn the attention of researchers because of the challenges in its transportation, storage and effective transformation. Solutions to these challenges are desirable because methane is an abundant resource that can be valorized as fuel and chemical feedstock. Moreover, environmentally conscience storage and transportation of methane is desired because of the strong greenhouse impact caused by the release of methane in the atmosphere.

Accordingly, the development of sustainable catalysis technologies for on-site methane liquefaction into valuable liquid fuels for the replacement of conventional off-site reforming processes has been an important research area in the chemical and energy industries, especially in the syngas-dependent methanol and formic acid syntheses. The direct and selective transformation of naturally abundant methane (CH₄) into high-value-added oxygenates, such as methanol, ethanol and formic acid, efficiently and cost effectively is desired. However, complex mixtures of products, often due to over-oxidation, make such transformations highly challenging, and difficult to perform efficiently and cost effectively.

The strategy of direct photocatalytic methane functionalization has been investigated as a potential alternative to circumvent issues such as requiring harsh reaction conditions, over-oxidation encountered in conventional thermal catalysis, and utilizing corrosive electrolyte in electro-catalysis. In the current methane oxidation systems with oxygen and water as oxidants, methane-activation mainly relies on the assistance of photo excited reactive oxygenated radicals (·OOH or ·OH) for cleaving C—H bond. As expected, excessive oxidative species used for achieving high productivity unavoidably makes complicated and uncontrollable mixture of oxygenated products. Therefore, realizing highly selective photo-conversion of methane into specific and useful oxygenated products remains a great challenge.

Many different semiconductors such as TiO₂, V/SiO₂, MoO₃, ZnO, WO₃, BiVO₄, C₃N₄, have been investigated as catalysts for the partial photo-oxidations of methane with nitric oxide (NO), hydrogen peroxide (H₂O₂), and even molecular oxygen (O₂) as oxidants to generate commodity oxygenates such as methanol, ethanol, and formic acid. To improve the photocatalytic efficiency of bare semiconductors, the loading of expensive noble metals (Au, Pd, Pt) or even binary metal (Au—Cu) was added on typical semiconductor-supports to enable the conversion of methane into C1 oxygenates. Unfortunately, all current photocatalytic semiconductors are still confronted with the inability to effectively control the production of a specific liquid oxygenated product and are also limited by the inability to sufficiently reduce or suppress over-oxidation thereby producing CO₂ and other over-oxidation byproducts.

Therefore, improvements in the conversion of methane to valuable oxygenated products are still desired particularly with respect to reducing or eliminating over-oxidation and to preferably be able to select for a specific oxygenated product.

### SUMMARY

There is provided a method of producing C1 oxygenated products comprising: providing an aqueous phase and GaN in a reactor having a closed environment; introducing from 0.1 to 5 bar of methane and from 0 to 10 bar of oxygen in the reactor; and irradiating the reactor with ultra-violet light until C1 oxygenated products are obtained. The C1 oxygenated products are preferably methanol and/or formic acid. The method can further comprise separating the C1 oxygenated products, such as methanol and/or formic acid.

In some embodiments, the closed environment of the reactor is evacuated before the step of introducing the methane and the oxygen, preferably to a pressure of less than 0.001 bar. In some embodiments, the closed environment of the reactor is purged with methane and optionally oxygen.

In some embodiments, the temperature during the step of irradiating the reactor is from 20 to 55° C. In some embodiments, the ultra-violet light has a wavelength of from 200 to 410 nm. In some embodiments, the aqueous phase is an aqueous suspension comprising the GaN. In some embodiments, the aqueous suspension comprises from 5 to 30 g/L of the GaN. In some embodiments, the GaN is supported on a zeolite or a silica gel and the reactor is a flow reactor.

In some embodiments, the C1 oxygenated products comprise at least 85% by weight of methanol. In some embodiments, the C1 oxygenated products comprise at least 70% by weight forming acid. To select for methanol, less than 0.1 bar of oxygen is introduced in the reactor. Preferably, no oxygen is introduced in the reactor. To select for forming acid, at least 0.35 bar of oxygen is introduced in the reactor.

Many further features and combinations thereof concerning the present improvements will appear to those skilled in the art following a reading of the instant disclosure.

### DESCRIPTION OF THE DRAWINGS

FIG. 1A is schematic representation of a photo-induced conversion of CH₄ to CH₃OH with GaN catalyst showing reduction reactions (R) and oxidation reactions (O).

FIG. 1B is schematic representation of a photo-induced conversion of CH₄ to HCOOH with GaN catalyst showing reduction reactions (R) and oxidation reactions (O).

FIG. 2A is a X-ray diffraction (XRD) pattern of commercial GaN semiconductor and standard GaN.

FIG. 2B is a transmission electron microscopy (TEM) image of GaN powder (scale bar 200 nm).

FIG. 2C is a TEM image of polycrystalline GaN powder showing its lattice structure (distance 5.18 A between the arrows).

FIG. 2D is a TEM image of polycrystalline GaN powder showing its lattice structure (distance 2.79 A between the arrows).

FIG. 2E is a photoluminescence (PL) spectrum of GaN powder.

FIG. 2F is a UV-Vis spectra with diffuse reflectance.

FIG. 2G is a Tauc plot produced from the UV-vis of FIG. 2F.

FIG. 2H is a X-ray photoelectron spectroscopy (XPS) valence band spectra.

FIG. 2I is a graph showing the electronic band alignment.

FIG. 2J is a graph of the UV-vis spectra of standard HCHO samples (concentrations 1.0 mM, 0.8 mM, 0.6 mM, 0.4 mM, 0.2 mM and base line).

FIG. 2K is a calibration curve of HCHO concentration by UV-vis spectra (the equation for the calibration is y=0.5581x with R²=0.9968).

FIG. 3A is a bar graph showing the yields of products after 20 h in conditions (i) without adding GaN catalyst, (ii) without light, (iii) with CH₃CN as solvent or (iv) under Ar reaction conditions.

FIG. 3B is a ¹H NMR spectra of the liquid products over 20 h with GaN for selective O₂-promoted methane photo-oxidation.

FIG. 3C is a ¹H NMR spectra of the liquid products over 20 h with GaN for selective O₂-free methane photo-oxidation.

FIG. 3D is a ¹H NMR spectra of the liquid products over 20 h reaction without GaN photo-catalyst.

FIG. 3E is a ¹H NMR spectra of the liquid products over 20 h reaction without light irradiation.

FIG. 3F is a gas chromatography-thermal conductivity detection (GC-TCD) spectra of the gas products after 20 h reaction without adding GaN catalyst.

FIG. 3G is a GC-TCD spectra of standard CO₂ samples (40 μmol, 32 μmol, 24 μmol, 16 μmol, and 8 μmol).

FIG. 3H is a GC-TCD calibration curve for the CO₂ concentration (calibration equation y=84990x with R²=0.9771).

FIG. 3I is a bar graph showing the yields of products after 20 h with GaN in the presence of 8 mL O₂ (labeled herein as GaN-802) or in the absence of O₂ (labeled herein as GaN-002).

FIG. 3J is a ¹³C NMR spectra of the liquid products after 20 h with GaN.

FIG. 3K is bar graph showing the yields of products after 20 h with GaN and Ga₂O₃ for O₂-free catalysis (methane photo-oxidation), i.e. 0 mL O₂ (labeled GaN-002 and Ga₂O₃-0O₂ respectively).

FIG. 3L is bar graph showing the yields of products over 20 h with GaN and Ga₂O₃ for O₂-promoted catalysis (methane photo-oxidation), i.e. 8 mL O₂ (labeled GaN-8O₂ and Ga₂O₃-8O₂ respectively).

FIG. 3M is a GC-TCD spectra of the gas products over 20 h with GaN (top line) and Ga₂O₃ (bottom line) for selective O₂-promoted methane photo-oxidation.

FIG. 3N is a GC-TCD spectra of the gas products over 20 h with GaN (top line) and Ga₂O₃ (bottom line) for selective O₂-free methane photo-oxidation.

FIG. 4A is a graph showing the productivity and selectivity towards formic acid of GaN with O₂ over different catalyst masses.

FIG. 4B is a GC-TCD spectra of the gas products after 2 h reaction with 100 mg of photo-catalyst.

FIG. 4C is a combined bar graph of the products yield and a graph of the selectivity for HCOOH in function of the reaction time.

FIG. 4D is a graph showing the yield in function of the number of photo-oxidation cycles the catalyst underwent.

FIG. 4E is a combined bar graph showing of the products yield and a graph showing the selectivity for methanol in function of the reaction time for various amounts of oxygen (0, 2, 4, 6, or 8 mL).

FIG. 4F is a combined bar graph showing the products yield and a graph showing the selectivity for methanol in function of the reaction time.

FIG. 4G is a bar graph showing the yields of products after 20 h of photo-oxidation with GaN, TiO₂, g-C₃N₄ or ZnO.

FIG. 4H is a GC-TCD spectra of the gas products after 20 h of photo-oxidation with g-C₃N₄.

FIG. 4I shows Arrhenius plots and the calculated activation energy for the O₂-promoted methane photo-oxidation of GaN (dotted line) and g-C₃N₄ (solid line).

FIG. 5A is a bar graph showing the yields of products after 20 h of photo-oxidation with GaN for selective O₂-promoted and O₂-free methane photooxidation with adding H₂O₂.

FIG. 5B is a UV-vis spectra of standard H₂O₂ samples (concentrations of 0, 0.96, 1.92, 2.88, 3.84, and 4.8 g/mL).

FIG. 5C is a UV-vis spectra calibration curve for the H₂O₂ concentration (calibration equation y=0.03115 x+0.00045 with R² 0.99899).

FIG. 5D is a graph showing the absorbance of nitroblue tetrazolium chloride in O₂-promoted photo-degradation in function of the wavelength and at different times (0 min, 5 min, and 10 min and for a blank sample).

FIG. 5E is a bar graph showing the photocatalytic H₂O₂ production of FIG. 5D.

FIG. 5F is an electron paramagnetic resonance (EPR) spectra of 5,5-dimethyl-1-pyrroline N-oxide (DMPO) adduct.

FIG. 5G is an EPR spectra of DMPO—OOH over GaN for selective O₂-free methane photo-oxidation.

FIG. 5H is a bar graph showing the turn over frequency (TOF) value of HCOOH over GaN for selective O₂-promoted (GaN-8O₂) and O₂-free (GaN-0O₂) methane photo-oxidation.

FIG. 5I is an EPR spectra of DMPO—CH₃ (♦) and DMPO—OH (▪) under O₂-promoted (top line) and O₂-free (bottom line) photocatalytic conditions.

FIG. 5J is a fluorescence spectra of 2-hydroxyterephthalic acid for hydroxyl radical measurement with GaN under different reaction parameters (no light (*), GaN-0O₂ (**) or GaN-8O₂ (***)).

FIG. 5K is a bar graph showing the TOF value of CH₃OH over GaN for selective O₂-promoted and O₂-free methane photo-oxidation.

FIG. 6A is a gas chromatography with a flame ionization detector (GC-ID) spectra of reaction products obtained in the absence of catalyst and light (negative control).

FIG. 6B is a GC-FID spectra of the reaction products obtained under the following reaction conditions: 100 mg of photocatalyst, 10 mL of H₂O, 0.7 bar of O₂, 0.3 bar CH₄, 25° C., 300 W Xe lamp.

FIG. 7 is a XRD pattern comparing a fresh GaN and a GaN who has undergone 6 reaction cycles.

FIG. 8 is a high resolution XPS measuring the Ga 3d spectra of GaN (peak).

### DETAILED DESCRIPTION

There is provided a method of producing C1 oxygenated products, preferably formic acid and methanol. Gallium nitride (GaN) is used as a photo-catalyst in an aqueous phase (e.g. suspended in the aqueous phase) or in contact with an aqueous phase (e.g. in a flow reactor set up) to obtain the C1 oxygenated products from methane. The reaction is performed in a reactor with a closed environment and having from 0.1 to 5 bar of methane and from 0 to 10 bar of oxygen in the reactor introduced therein. The GaN catalyst is activated by irradiation with ultra-violet (UV) light. In some embodiments, the method can selectively produce methanol or selectively produce formic acid. The selectivity towards methanol is controlled by having limited oxygen or an oxygen-free environment in the reactor, for example less than 0.1 bar of oxygen, and the selectivity towards formic acid is controlled by having an oxygenated environment in the reactor, for example at least 0.35 bar of oxygen.

GaN is a III-V nitride semiconductor with a d¹⁰ electronic configuration. The very high bonding energy (8.9 eV/atom) of the GaN bond with a largely ionic component character, makes GaN a thermally and chemically stable material with an ultrahigh melting point (>2500° C.). GaN is resistant to decomposition up to at least 1000° C., even under vacuum.

GaN is a methane-active semiconductor that catalyzes the photo-oxidation of methane and empowers fine-controlling of chemo-selectivity towards methanol or formic acid, simply by regulating the O₂ content in the aqueous phase. The aqueous phase can be defined as a composition comprising at least 75 wt. % of atomic water (H₂O), preferably at least 80 wt. % or at least 85 wt. %. The aqueous phase can be a suspension and the solvent of the suspension can be water, deionized water, saline water or an aqueous buffer. Indeed, by providing oxygen gas in the reactor environment, at least a portion of the gaseous oxygen dissolves or penetrates into the aqueous phase. On the other hand, by not introducing any gaseous oxygen in the reactor, the oxygen content in the aqueous phase is limited or eliminated.

GaN is characterized by a regular wurtzite crystal structure, which is the thermodynamically stable phase of GaN. In this crystal structure, the exposed surfaces of the GaN nanoparticles are composed of c-planes and m-planes. The m-plane is a one dimensional rectangular configuration of the Ga and N atoms and the c-plane is a one dimensional hexagonal configuration of the Ga and N atoms. The overall m-plane of GaN is nonpolar since it is composed of equal numbers of Ga and N atoms which are tetrahedrally coordinated with each other, whereas the polar c-plane comprises only one type of atom (either Ga or N) which exhibits piezoelectric polarization along the c-axis.

In some embodiments, the GaN is in the form of a powder which may have a grain size of from 100 to 500 Å, from 150 to 350 Å or from 200 to 250 Å. In other embodiments, the GaN is in the form of a nanoparticle which is defined as having a diameter in the nanoscale. For example, the GaN nanoparticles can have a diameter of between 10 and 1000 nm, between 20 and 900 nm, between 30 and 800 nm, or between 40 and 700 nm. In further embodiments, the GaN is supported by a catalyst support, for example zeolite or silica gel solid supports. At industrial scale the use of a flow reactor is generally more cost effective than a batch system, accordingly, it is preferred to have the catalyst on a solid support rather than in suspension.

In some embodiments, the GaN has a purity of at least 90%, at least 95%, at least 98%, or at least 99%. In some embodiments, the GaN can be a doped GaN and the dopant can be selected from Pt, Pd, Au, Ag or combinations thereof such as an Au—Pd bimetal center. Dopants can advantageously improve the reactivity of the catalyst.

In contrast to previous methods, limited/reduced or preferably no over-oxidation products (such as CO₂ and CO) are produced by the present process. The present method achieves highly selective reactivity and tunability thanks to the controlled generation of moderately reactive oxygen radicals (such as ·OOH and ·OH) in combination with the direct methane activation triggered by GaN through a photo-generated radical process. The moderately reactive radicals of ·OOH and ·OH are generated in a controllable manner triggered by GaN-semiconductor under UV irradiation, which is primarily responsible for tuning the selectivity towards different C1 oxygenated products through the sophisticated methane oxidation.

Herein, a dual selective conversion of methane into formic acid and methanol with molecular oxygen and water as oxidants, respectively, under ambient conditions in water was accomplished by using the GaN semiconductor. In the photocatalytic system of the present disclosure, GaN, owing to its unique photo-physical property and methane-activation ability, achieves the specific production of methanol with at least 90% selectivity or formic acid with at least 80% selectivity, respectively, by controlling the O₂ content in water. Namely, the primary product is methanol under O₂-free conditions and formic acid in the presence of O₂. The moderately reactive radicals of ·OOH and ·OH are generated in a controllable manner triggered by the GaN-semiconductor under ultra-violet (UV) irradiation, which is primarily responsible for tuning the selectivity towards different C1 products in sophisticated methane oxidation.

Accordingly, one advantage of the present method is the reduction or elimination of the production of over-oxidation products such as carbon dioxide and carbon monoxide. Carbon dioxide and carbon monoxide are undesirable by-products that are harmful for the environment. Moreover, the generation of over-oxidation products reduces the yield of the desired oxygenates and therefore, an improved yield is obtained by minimizing or eliminating the production of over-oxidation products. In some embodiments, the products mixture obtained by the present method contains less than 3 wt. %, less than 2 wt. %, less than 1 wt. %, less than 0.5 wt. % or less than 0.1 wt. % of total over-oxidation products such as carbon dioxide and carbon monoxide.

In preferred embodiments, the closed environment of the reactor is evacuated to a pressure of less than 0.001 bar, less than 10⁻⁴ bar, less than 10⁻⁵ bar, less than less than 10⁻⁶ bar or preferably 0 bar. The reactor can therefore be an air-tight reactor or a hermetically sealed reactor. The evacuation can be performed before or after providing the aqueous phase containing the GaN into the closed environment. The evacuation can be performed by any suitable vacuum pump such as an oil pump (vacuum pump). After the evacuation, the remaining gaseous species can be trace amounts of air, which generally are negligible. In some cases where air is initially in the reactor, then the reactor contains an initial amount of oxygen. This oxygen is preferably evacuated in order to then introduce a precise and controlled volume of oxygen gas or in some embodiments obtain oxygen free reaction conditions. In some embodiments, in addition or alternatively to the evacuation, the reactor is purged with methane and optionally oxygen. Alternatively or in addition to the evacuation, the closed environment can be purged with methane and optionally oxygen. For example, in order to selectively produce methanol, the purging can be done with methane only. On the other hand, in order to selectively produce formic acid, the purging can be done with methane and oxygen.

An aqueous phase and GaN are provided in the reactor. In some embodiments, the aqueous phase is an aqueous suspension comprising from 5 to 30 g/L, from 10 to 30 g/L, from 10 to 25 g/L, or from 10 to 20 g/L of the GaN. In other embodiments, the GaN can be supported on a solid support for example on a wall of a flow reactor, and the GaN is brought into contact with the aqueous phase by flowing the aqueous phase on the solid support. The aqueous phase or the aqueous suspension is water, preferably deionized water.

In some embodiments, to perform the evacuation after adding the aqueous phase, the reactor is subjected to a freezing step, for example by using liquid nitrogen. The freezing step allows to solidify the aqueous phase so that it is not evacuated by the vacuum pump. In some embodiments, an evacuation step can be performed before and/or after introducing the aqueous phase in the reactor.

Methane and oxygen are introduced in the reactor so that the pressure of methane in the reactor is from 0.1 to 5 bar and the pressure of oxygen in the reactor is from 0 to 10 bar. In some embodiments, the methane gas pressure in the reactor is from 0.1 to 4 bar, 0.1 to 3 bar, 0.1 to 2 bar, 0.1 to 1 bar, 0.1 to 0.8 bar, 0.1 to 0.7 bar, 0.1 to 0.5 bar, or 0.2 to 0.4 bar, and preferably 0.3 bar. As previously explained, the oxygen content in the reactor drives the selectivity of the photo-catalysis to methanol in minimal oxygen conditions or in the absence of oxygen, or to formic acid in an oxygen containing environment (e.g. excess of oxygen). The pressure of oxygen in the reactor can be up to 10 bar, up to 9 bar, up to 8 bar, up to 7 bar, up to 6 bar, up to 5 bar, up to 4 bar, up to 3 bar, up to 2 bar, or up to 1 bar.

The switching of selectivity from formic acid to methanol can be achieved by gradually decreasing the amount of O₂ in the GaN-catalyzed photo-conversion of methane in water. In some embodiments, to select for methanol, the reactor can contain less than 0.1 bar, less than 0.05 bar, less than 0.03 bar, less than 0.01 bar or be free of O₂. The expression “select for methanol” is defined as achieving a selectivity of at least 85 wt. %, at least 87.5 wt. %, at least 90 wt. % or more of methanol with respect to the total weight of the products obtained. In some embodiments, to select for forming acid, at least 0.35 bar, at least 0.4 bar, at least 0.45 bar, at least 0.5 bar, at least 0.525 bar, at least 0.6 bar, from 0.35 to 1 bar, from 0.35 to 0.9 bar, from 0.35 to 0.8 bar, from 0.35 to 0.7 bar, from 0.4 to 0.65 bar, from 0.45 to 0.6 bar or from 0.5 to 0.55 bar of oxygen is present in the reactor during the irradiation. The expression “select for formic acid” can be defined as achieving a selectivity of at least 70 wt. %, at least 72.5 wt. %, at least 75 wt. %, at least 77.5 wt. %, at least 79 wt. %, at least 80 wt. %, at least 82.5 wt. % or at least 85 wt. % or more of formic acid with respect to the total weight of the products obtained.

With O₂, more pronounced ·OOH radical are obtained and there is an O₂-promoted in situ generation of ·OOH radical, which are enriched on the surface of GaN. This has a positive influence on pushing the catalytic process towards the synthesis of deep oxidation products (i.e. formic acid) while still avoiding over-oxidation to CO₂.

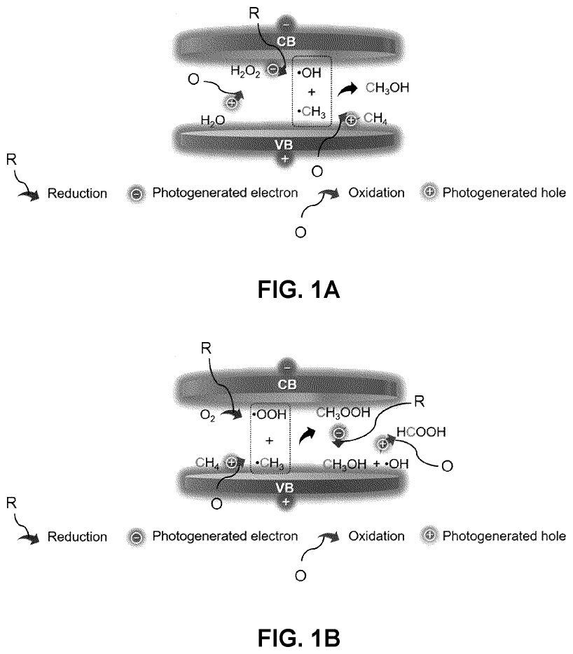

Without wishing to be bound by theory, the present disclosure proposes a mechanism for the dual selective photocatalytic CH₄ oxidation to formic acid and methanol under O₂ and O₂-free conditions catalyzed by GaN as shown in FIGS. 1A and 1B. In the O₂-free condition illustrated in FIG. 1A, initial H₂O photo-oxidation occurs at photo-generated holes of GaN to form H₂O₂. Subsequently, ·OH and ·OOH, derived from H₂O₂ decomposition, react with ·CH₃ generated on GaN to form methanol. When oxygen is provided (FIG. 1B), the extra O₂ undergoes reduction on GaN to produce excess ·OOH and ·OH radicals, which further oxidize methanol into formic acid.

As can be seen in FIGS. 1A and 1B, in situ H₂O₂ is produced at the catalyst surface. The present methane photo-conversion thus undergoes a hydroperoxyl event (i.e. a hydroperoxyl radical is produced). The external addition of H₂O₂ in the present process does not result in an increase in yield. In fact, a notable drop in total yield of total liquid oxygenates and selectivity to either desired formic acid or methanol was observed. This establishes the irreplaceable role of in situ generated H₂O₂ on the GaN surface, rather than external H₂O₂, for improving the selective generation of methanol and formic acid. Accordingly, in some embodiments, the present method is free of any H₂O₂ external additions. In other words there is no deliberate addition of H₂O₂ before the reaction begins.

To initiate the catalysis by GaN, the reactor is irradiated to expose the UV-sensitive GaN to UV light. The GaN is able to grant easy access to methyl radical for the oxygen species via the direct methane activation due to its strong oxidizing ability. The UV light can have a wavelength of from 200 to 410 nm or from 300 to 410 nm. GaN semiconductor, is a prospective photosensitizer with sufficient redox capacity that enables a compromise between activation energy barriers of both two-electron water oxidation and O₂ reduction to form H₂O₂ and related oxygen species in situ for facilitating the subsequent indirect methane activation. The irradiation can be performed for a length of time sufficient to obtain the desired C1 oxygenates such as methanol or forming acid. In some cases, the irradiation can last at least 2 h, at least 3 h, at least 4 h, at least 5 h, at least 10 h, at least 15 h, at least 20 h or more as needed. During the irradiation and reaction, the reactor can be maintained at room temperature by submerging the reactor in a chiller. Room temperature is defined as temperatures in the range of 20-55° C. or 20-30° C., for example 25° C. The reaction is preferably maintained at a temperature below 55° C., below 50° C., below 40° C., or below 30° C. in order to reduce the production of by-products (e.g. carbon oxygenated compounds other than methanol and formic acid).

The C1 oxygenated products (e.g. methanol and formic acid) can each be separated from the product mixture after irradiation. Separation techniques including solvent extraction and distillation can be used. The GaN catalyst can also be separated and reused in further oxidation cycles. The GaN catalyst can be separated by any suitable separation technique such as centrifugation or filtration. One advantage of the GaN catalyst is that the catalyst can be reused in order to save cost in additional oxidation reactions. For example, the GaN catalyst can be used for a total of 2, 3, 4, 5, 6 cycles or more.

The present disclosure has achieved a dual selective photoconversion of CH₄ to HCOOH or CH₃OH via GaN-catalysis with or without O₂ in water. Photo-excited holes at the GaN surface exhibited powerful oxidizing capacity to directly activate the methane C—H bond in the absence of O₂ for generating CH₃OH in an excellent selectivity of at least 90% for example. Furthermore, the enhanced generation of oxygen radical species such as ·OH and ·OOH in the presence of O₂ is established to drive the continuous oxidation of methanol into HCOOH with a selectivity of at least 79%, for example. Such a selective tunability towards specific oxygenated products based on GaN-semiconductor not only leads to practical application of methane and natural gas utilizations in the chemical and energy sectors, but also allows for the development of semiconductor-prompted functionalization of C—H bonds such as in late-stage functionalization of pharmaceuticals and other organic materials.

### EXAMPLE

The following materials were purchased from suppliers: commercial GaN catalyst (99.9% purity) was purchased from Alfa Aesar™ and used without further treatment; methane (99.99% purity) was purchased from Air Liquide™. ¹³CH₄ (99 atom % ¹³C) was purchased from Sigma-Aldrich™; oxygen (99.99% purity) was purchased from Praxair™; and commercially available semiconductors (Ga₂O₃, TiO₂, ZnO, C₃N₄) were purchased from Sigma-Aldrich™ and used without further purification.

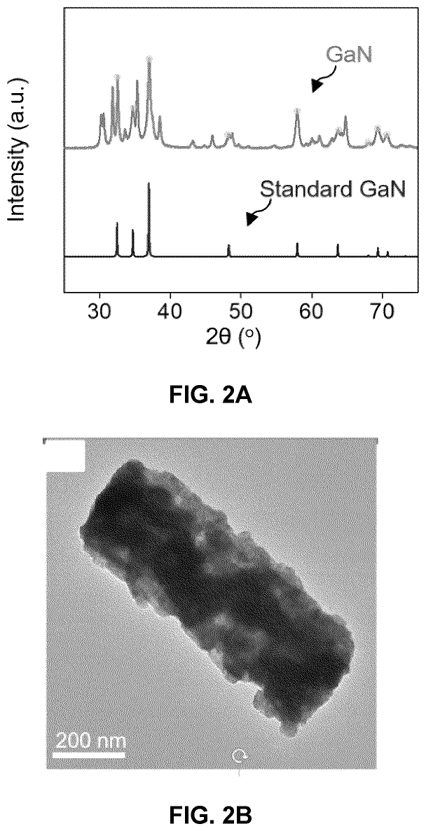

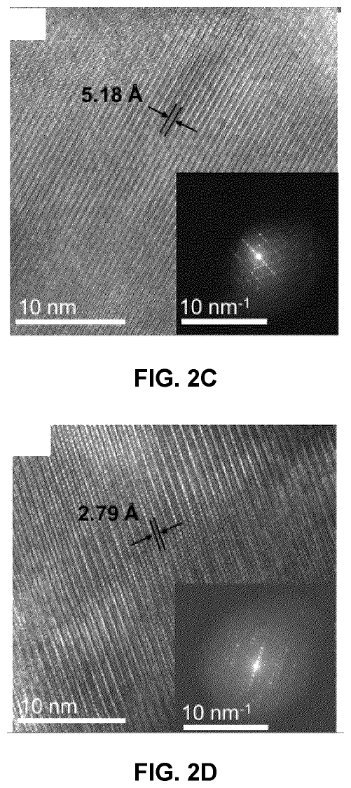

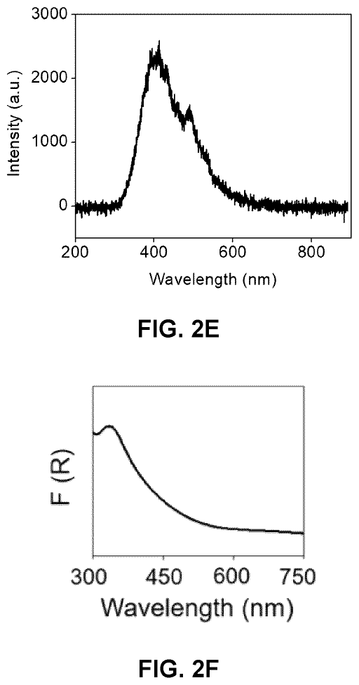

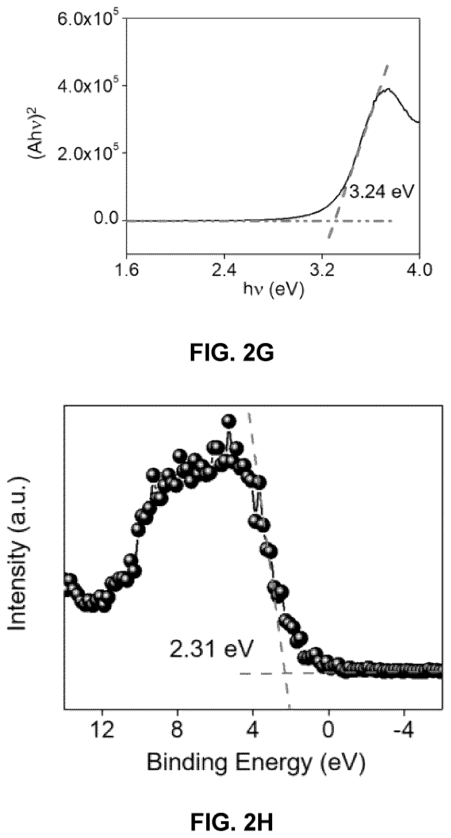

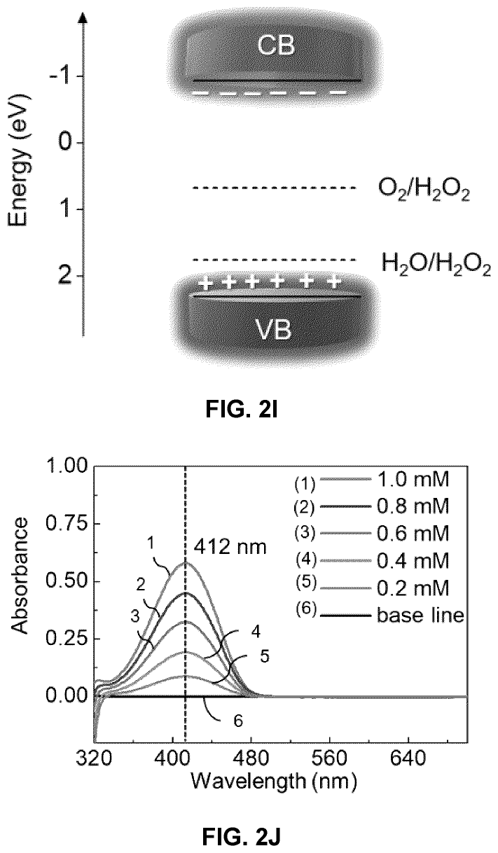

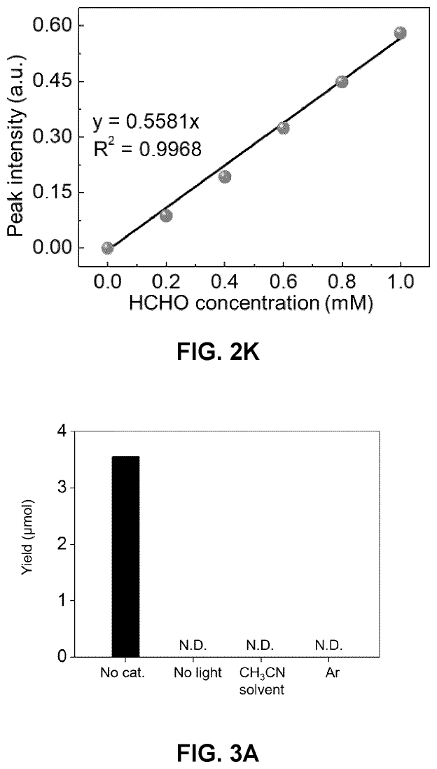

The GaN purchased was a pale-yellowish powder which was characterized to determine its chemostability and its semiconducting properties. A powder X-ray diffraction (XRD) analysis was performed on the GaN and XRD patterns were obtained with a Bruker™ DD8 Advanced diffractometer with Cu Kα radiation (λ=1.5418 Å). 10 mg of GaN powder was loaded into a low-background sample holder to perform the XRD analysis. The instrument was operated with an increment of 0.02° and a counting time of 24 s under the voltage of 40 kV and 40 mA. FIG. 2A shows the XRD pattern reflecting a typical wurtzite crystalline structure of the GaN. The GaN was imaged by bright field transmission electron microscopy (TEM) with a FEI Tecnai™ G2 F20 S/TEM at accelerating voltage of 200 kV. Firstly, 1 mg of GaN powder was dispersed in 1.5 mL ethanol solution to obtain a diluted solution. Then, 10 μL of the diluted solution was added dropwise into the super thin carbon sample holder, it was dried at room temperature, and then placed into the TEM microscope for observation. The high-resolution transmission electron microscopy (HRTEM) and their corresponding electron diffraction patterns further confirm that the polycrystalline GaN powder is mainly composed of the lattice fringe of c-planes (0001) and m-planes (1100) (FIGS. 2B-2D). A photoluminescence (PL) measurement was performed with either a 405-nm laser or a 325-nm He—Cd laser (Kimmon Koha) as excitation source. 1 mg of GaN powder was placed on a carbon tape, and the sample was pressed to form a film. The film was then placed on the center of the sample holder for instrument testing. As observed by PL (spectrum shown in FIG. 2E), the commercial GaN semiconductor had an ultraviolent light absorption from 300 nm to 410 nm with a corresponding band gap of 3.24 eV calculated from the diffuse reflectance (DR) spectra measured by UV-vis spectrometry, performed on Cary™ 5000 UV-Vis-NIR Spectrophotometer from Agilent™ (FIG. 2F), and from the corresponding Tauc plot obtained with the DR (FIG. 2G).

The GaN was also analyzed by X-ray photoelectron spectroscopy (XPS) which was conducted on an ESCALAB™ 250 X-ray photoelectron spectrometer with a monochromated X-ray source (Al Kα hv=1486.6 eV), and the energy calibration of the spectrometer was performed using C 1 s peak at 284.8 eV. Briefly, 1 mg GaN powder was placed on the copper foil of photoelectron spectrometer, which was then evacuated to perform the test. Combining with the X-ray photoelectron spectroscopy (XPS) valence band spectra (FIG. 2H), semiconducting band alignment of commercial GaN was identified and shown in FIG. 2I where the two-electron water oxidation potential and O₂ reduction potential to H₂O₂ locates between the top of valance band (2.31 eV) and the bottom of conduction band (−0.93 eV). Hence, GaN semiconductor, is a photosensitizer with sufficient redox capacity as it was shown to enable a compromise between the activation energy barriers of both two-electron water oxidation and O₂ reduction to form H₂O₂ and related oxygen species in situ for facilitating the subsequent indirect methane activation. Table 1 below, summarizes the oxidations and reductions relating to O₂, H₂O and H₂O₂ with GaN.

TABLE 1
Summary of standard potentials related to O₂, H₂O and
H₂O₂ of E vs RHE with E being the equilibrium potential
of the half reaction, and RHE being the reversible hydrogen
electrode (RHE), which is a reference electrode for the
measurement of other half reaction potential.

| Half reaction | E vs. RHE |
|---|---|
| O₂ + 2H⁺ + 2e⁻ → H₂O₂ | +0.68 V |
| 2H₂O + 2h⁺ → H₂O₂ + 2H⁺ | +1.76 V |
| H₂O₂ + H⁺ + 2e⁻ → •OH + H₂O | +0.38 V |

The challenging polarization and cleavage of the C—H bond of a CH₄ molecule is promoted by GaN-based materials and it is demonstrated below that GaN can be leveraged to catalyze the production of methanol and formic acid.

The GaN catalyst was first evacuated at 250° C. for 2 h to remove water and other molecules adsorbed in the powders. In the CH₄ photocatalytic oxidation process, a suspension of deionized water (1 mL) with the corresponding amount of evacuated GaN powder (10-40 mg) was added to an air-tight quartz reactor (12 mL quartz tube). The reactor was then completely evacuated by oil pump (vacuum pump) after being frozen by liquid nitrogen (N_{2(liquid)} at 196° C. for 10 s), followed by the introduction of 3 mL CH₄ gas (0.3 bar) and corresponding amount of O₂ gas (0-8 mL, 0-0.7 bar) with syringes under room temperature. Afterwards, the reactor was partially submerged in a 25° C. chiller and illuminated by a 300w full-arch Xe lamp (PE300 BUV) for 20 hours to complete the reaction. After the light irradiation (above 200 nm), the gas products were qualitatively analyzed by gas chromatograph (Agilent™ 6890N Network Gas Chromatograph) equipped with thermal conductivity detector (TCD). The liquid products were quantified by nuclear magnetic resonance (Bruker™ Ascend 1 500 MHz Spectrometer) spectroscopy, in which dimethyl sulfoxide (DMSO, Sigma-Aldrich, 99.99%) was added as an internal standard.

The photocatalytic performance of GaN for the CH₄ oxidation with O₂ and water was tested in a quartz tube reactor at room temperature (25° C.) under the irradiation of a 300 W Xenon lamp (>>200 nm) as explained above. In this experiment, the reaction conditions were: 1 mL of deionized water, 0.7 bar of O₂, 0.3 bar of CH₄ or Ar, 25° C., and irradiation (above 200 nm) with the 300 W Xe lamp.

The quantification of HCHO was performed by a colorimetric method as described in H. Song, X. Meng, S. Wang, W. Zhou, X. Wang, T. Kako, J. Ye, Direct and selective photocatalytic oxidation of CH₄ to oxygenates with O₂ on co-catalysts/ZnO at room temperature in water. J. Am. Chem. Soc. 141, 20507-20515 (2019). Briefly, 100 ml of reagent aqueous solution was first prepared by dissolving 15 g of ammonium acetate, 0.3 mL of acetic acid, and 0.2 mL of pentane-2,4-dione in water. Then, 0.5 mL of liquid product was mixed with 2.0 ml of water and 0.5 mL of reagent solution. The mixed solution was maintained at 35° C. for 1 hour and measured by UV-vis adsorption spectroscopy until the adsorption intensity at 412 nm did not further increase. The UV-vis calibration curves are shown in FIGS. 2J-2K.

For the quantification of the remaining liquid products (CH₃OH, HCOOH, CH₃COOH, HOCH₂OOH), ¹H nuclear magnetic resonance (NMR) (Bruker™ Ascend 1 500 MHZ Spectrometer) was used. Typically, 0.6 mL liquid product was mixed with 0.1 mL of D₂O, and 0.025 μL DMSO was added as an internal standard. The products were quantified by comparing the ¹H NMR signal of the products and the internal standard. The signal of protons from the solvent H₂O is much higher than that from the products. Therefore, all ¹H NMR spectra were recorded using a pre-saturation solvent suppression technique to suppress the dominant H₂O signal.

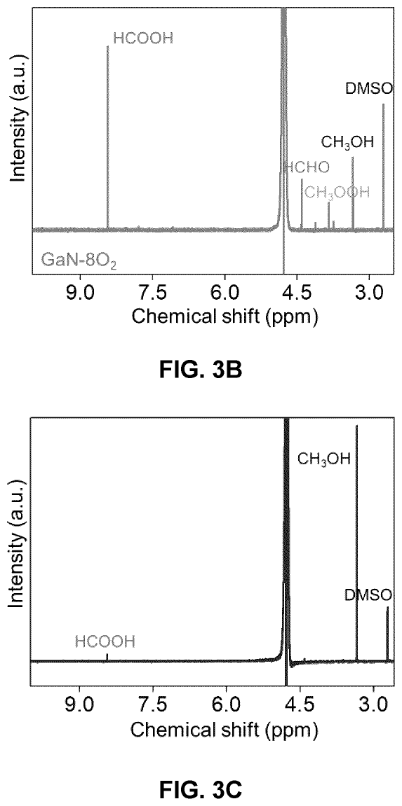

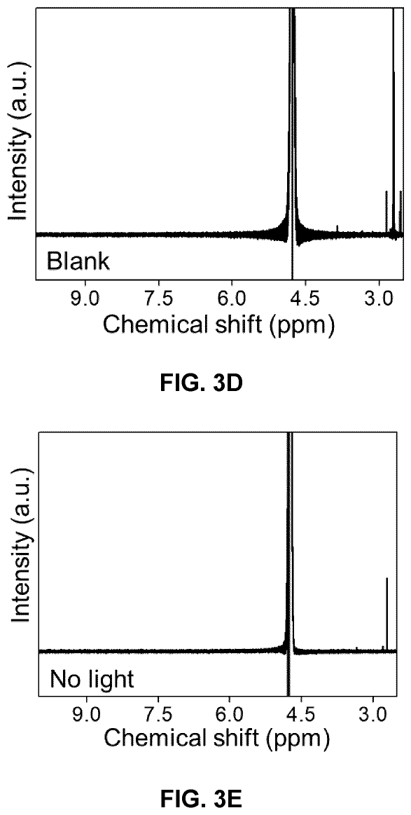

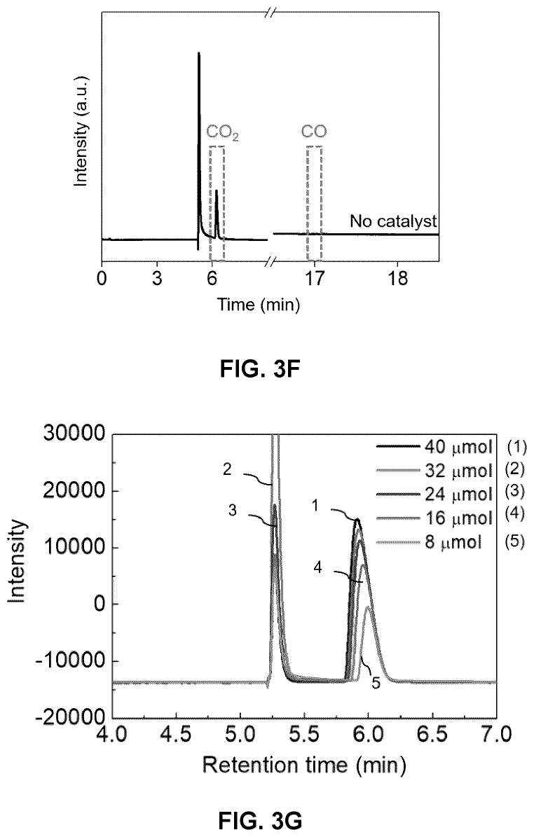

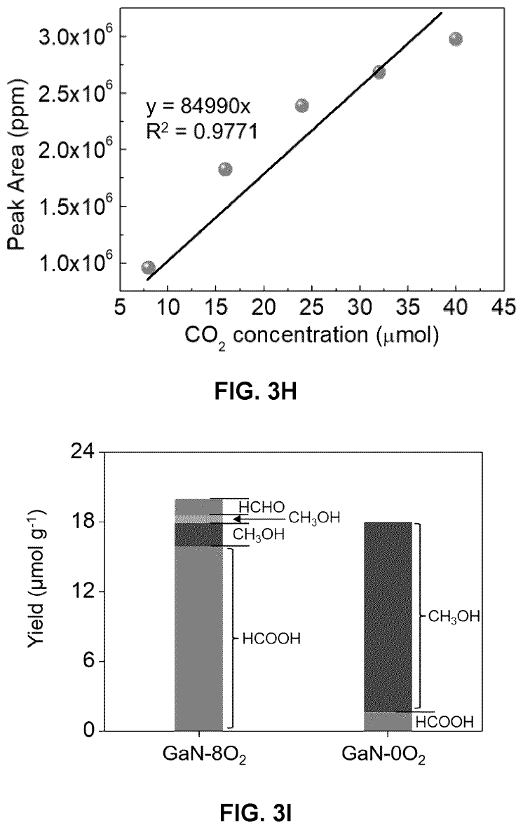

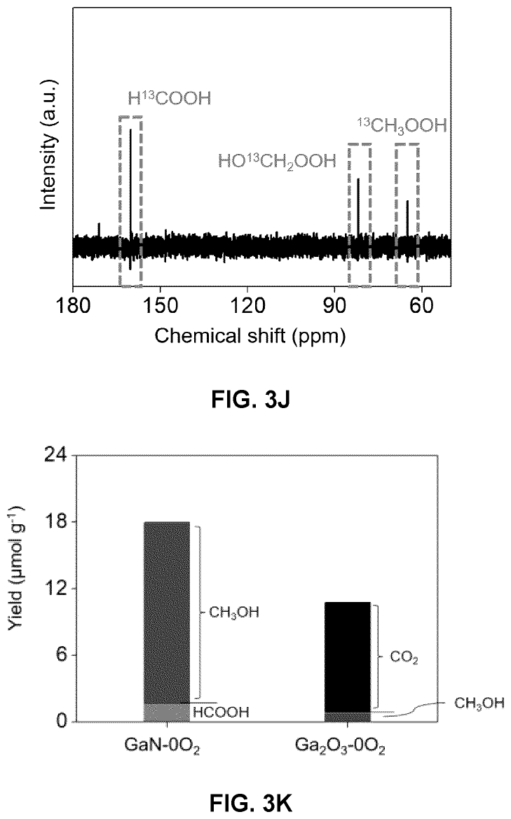

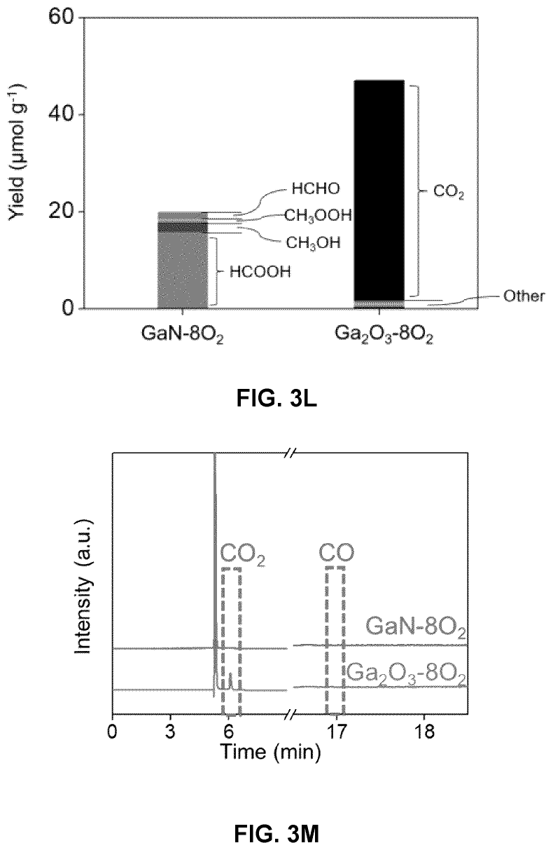

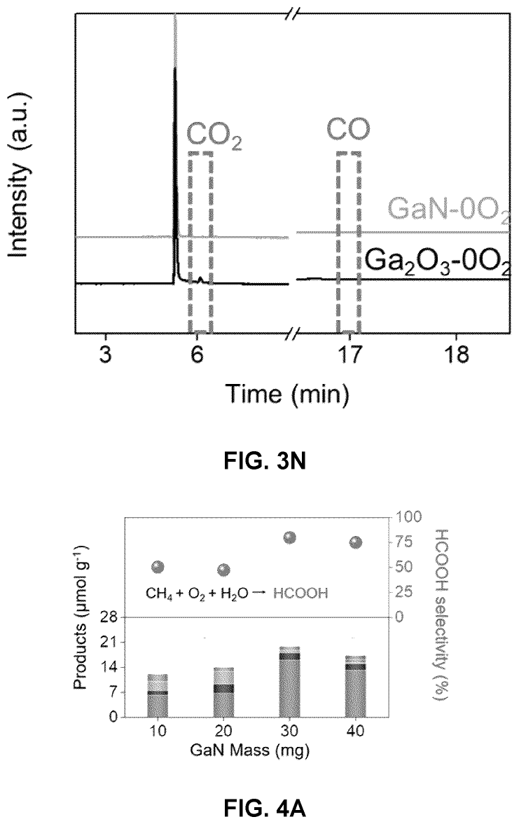

The conversion of methane to liquid oxygenated products did not take place in the absence of UV light, in the absence of methane or when using acetonitrile instead of water as solvent (FIG. 3A and Table 2). In FIG. 3A the yield for the no catalyst (no cat.) condition is entirely CO₂. The ¹H NMR spectra for the quantification are shown in FIGS. 3B-3E.

TABLE 2
The yields of products over 20 h without adding GaN
catalyst, without light, with CH₃CN as solvent and
under Ar reaction conditions. n.d. = not detected

| | Amount of products (μmol) | | | | |
|---|---|---|---|---|---|
| Samples | HCOOH | CH₃OH | CH₃OOH | HCHO | CO₂ |
| No | n.d. | trace | n.d. | n.d. | 3.56 |
| catalyst | | | | | |
| No light | n.d. | n.d. | n.d. | n.d. | n.d. |
| CH₃CN | n.d. | n.d. | n.d. | n.d. | n.d. |
| solvent | | | | | |
| Ar | n.d. | n.d. | n.d. | n.d. | n.d. |

Trace oxygenated mixtures and a large amount of CO₂ were observed under standard photocatalytic condition in which methane gas was added but in absence of the catalyst. In FIG. 3A the yield for the no catalyst (no cat.) condition is entirely CO₂.

FIG. 3F shows the gas chromatography—thermal conductivity detection (GC-TCD) spectra of the gas products after 20 h reaction without adding a GaN catalyst. Briefly, 1 mL of the gas sample was injected in to the GC-TCD. The temperature program of the oven was as follows: the starting temperature was 60° C., it was maintained for 10 min, then, the temperature was elevated to 280° C. at the rate of 120° C./min. The temperature was maintained at 280° C. for another 5 min. The reaction conditions were 0 mg of catalyst, 1 mL of H₂O, 8 mL of O₂ (0.7 bar), 3 mL (0.3 bar) of CH₄, 25° C., and irradiation by 300 W Xe lamp. The GC-TCD spectra calibration standard curves for CO₂ samples are shown in FIGS. 3G-3H.

The same experimental conditions were repeated while adding the GaN catalyst. Strikingly, the introduction of the GaN semiconductor resulted in the generation of liquid oxygenates in which the selectivity towards primarily C1 products reaches up to 99% in total, including methanol (9.79%), methyl peroxide (3.80%), formaldehyde (6.66%) and formic acid (79.75%) (FIG. 3I and Table 3).

TABLE 3
The yields and selectivity of products over 20 h with GaN for
selective O₂-promoted and O₂-free methane photo-oxidation.

| | O₂, | Yield, | Selectivity, % | | | | |
|---|---|---|---|---|---|---|---|
| Sample | bar | μmol/g | HCOOH | CH₃OH | CH₃OOH | HCHO | CO₂ |
| GaN | 0 | 17.96 | 9.35 | 90.65 | 0 | 0 | 0 |
| GaN | 0.7 | 20.01 | 79.75 | 9.79 | 3.80 | 6.66 | 0 |

To determine the origin of the carbon in the reaction products, the experiment was repeated with ¹³CH₄ instead of methane (¹³CH₄ isotopic experiment). The reaction conditions were: 30 mg GaN catalyst, 1 mL of H₂O, 8 mL of O₂ (0.7 bar), 0.3 bar of ¹³CH₄, 25° C., and 300 W Xe lamp irradiation. The reaction products of the ¹³CH₄ isotopic experiment were analyzed after 20 h by ¹³C NMR. The resulting NMR spectra is shown in FIG. 3J.

The production of C1 oxygenates with GaN catalyst was compared to the production with a Ga₂O₃ catalyst. The yields of the products were compared after 20 h with GaN or Ga₂O₃ for the selective O₂-promoted and O₂-free methane photo-oxidation. The reaction conditions were 30 mg catalyst (GaN or Ga₂₀₃), 1 mL of H₂O, 8 or 0 mL of O₂ (0.7 bar or 0 bar), 3 mL (0.3 bar) of CH₄, 25° C., and irradiation with the 300 W Xe lamp. The product yields are shown in FIGS. 3K-3L and in Table 4. The GC-TCD spectra of the gas products over 20 h with GaN and Ga₂O₃ were compared with O₂ (0.7 bar) or without (0 bar) (FIG. 3M-3N). As emphasized in FIGS. 3M-3N in the dotted box, the Ga₂O₃ catalyst produced CO₂ whereas the GaN catalyst did not, and both the Ga₂O₃ and the GaN did not produce CO. Throughout the experiments performed, negligible over-oxidation products such as CO₂ and CO were detected using GaN catalyst (FIGS. 3I, 3K-3N and Tables 3-4). All these results demonstrated that the GaN-semiconductor catalyzed photo-conversion of methane with O₂ into oxygenates proceeded in water in absence of over-oxidation.

TABLE 4
The yields and selectivity of products over 20 h with GaN and
Ga₂O₃ for selective O₂-promoted and O₂-free methane photo-oxidation.

| | O₂, | Yield, | Selectivity, % | | | | |
|---|---|---|---|---|---|---|---|
| Sample | bar | μmol/g | HCOOH | CH₃OH | CH₃OOH | HCHO | CO₂ |
| GaN | 0 | 17.96 | 9.35 | 90.65 | 0 | 0 | 0 |
| Ga₂O₃ | 0 | 10.76 | 0 | 7.12 | 0 | 1.21 | 91.67 |
| GaN | 0.7 | 20.01 | 79.75 | 9.79 | 3.80 | 6.66 | 0 |
| Ga₂O₃ | 0.7 | 47.74 | trace | 1.01 | 1.84 | 0.78 | 96.37 |

In theory, enhancing the exposure of active GaN surface with methane can increase both the C1 yield and the product selectivity by varying the catalyst loading. The experiment was performed with different catalyst amounts: 10, 20, 30 and 40 mg. The remaining reaction conditions were as follows: 8 mL O₂, 1 mL of H₂O, 120 μmol of CH₄ under 1 atm, 25° C., and irradiation by the 300 W Xe lamp. The product yield and the turn over frequency (TOF) were calculated according to the following equations:

$$Yield = \frac{( number of moles of ( HCOOH + {CH}_{3} OH + {CH}_{3} COOH + HCHO + {HOCH}_{2} OOH ) )}{weight of catalyst}$$

$$TOF = \frac{( number of moles of ( HCOOH + {CH}_{3} OH + {CH}_{3} COOH + HCHO + {HOCH}_{2} OOH ) )}{( weight of catalyst ) * ( reaction time )}$$

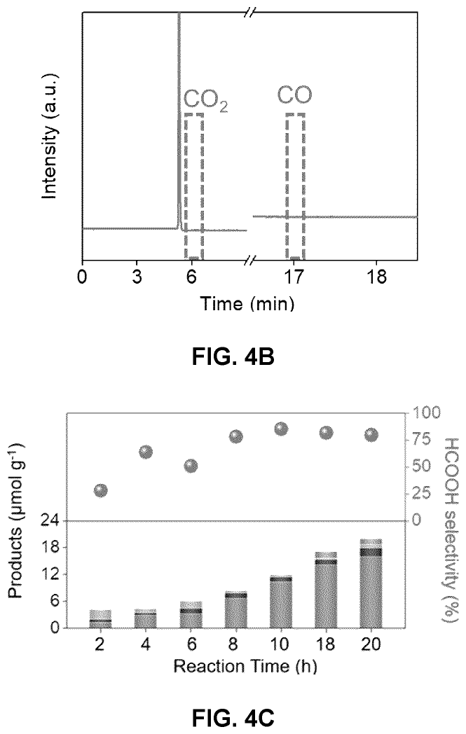

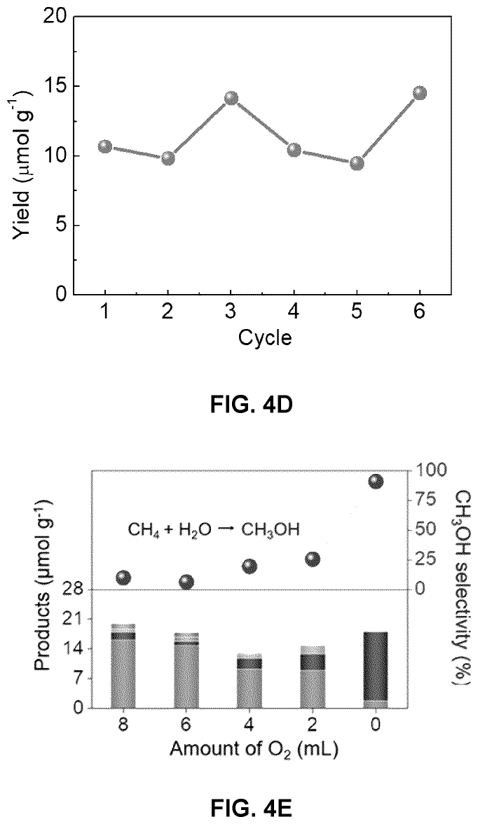

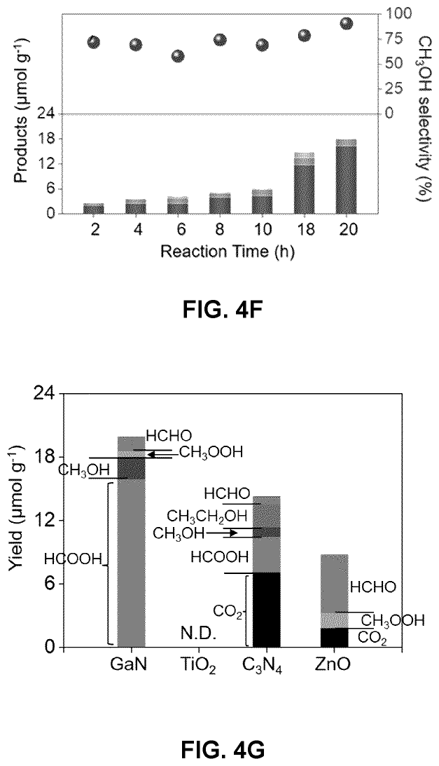

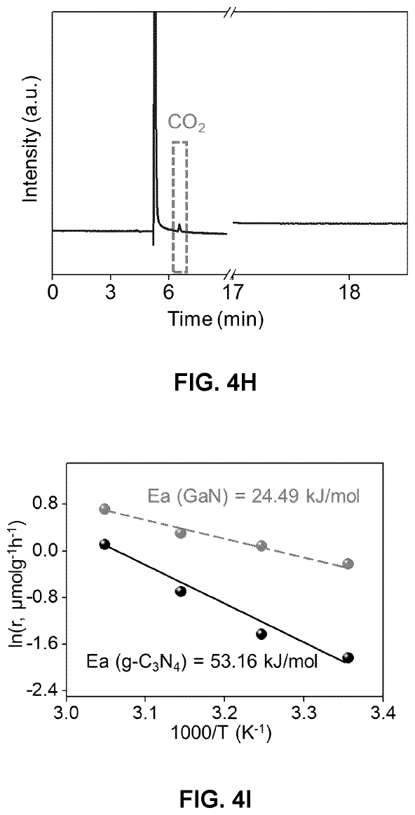

The total yield of C1 liquid oxygenates was increased to 20.01 μmol g⁻¹ cat from 12.16 μmol g⁻¹ cat when increasing the mass of GaN catalyst from 10 mg to 30 mg. Further increasing the GaN catalyst mass to 40 mg did not improve either the yield or selectivity, because of the blocking from extra sample to effective GaN surface (FIG. 4A, Table 5). The following reaction conditions were also tested: 100 mg of photocatalyst, 10 mL of H₂O, 0.7 bar of O₂, 0.3 bar CH₄, 25° C., 300 W Xe lamp (FIG. 4B). No CO or CO₂ was detected (FIG. 4B).

TABLE 5
The yield and selectivity of products over 20 h
with GaN with O₂ over different catalyst masses

| GaN, | Yield, | Selectivity, % | | | | |
|---|---|---|---|---|---|---|
| mg | μmol/g | HCOOH | CH₃OH | CH₃OOH | HCHO | HOCH₂OOH |
| 40 | 17.43 | 74.87 | 10.84 | 1.55 | 4.73 | 8.01 |
| 30 | 20.01 | 79.75 | 9.79 | 3.80 | 6.66 | 0 |
| 20 | 14.08 | 47.30 | 18.32 | 25.14 | 9.23 | 0 |
| 10 | 12.16 | 50.33 | 9.87 | 21.71 | 18.09 | 0 |

Likewise, the selectivity towards formic acid was also increased from 61.45% to 79.74% upon excitation for 8 h and was maintained afterwards (FIG. 4C and Table 6). Using such reaction conditions, an optimal yield (16.01 μmol g cat-1) and a high selectivity towards HCOOH (˜80%) could be obtained after 20 h.

TABLE 6
The time-dependent yield and selectivity under optimal GaN
mass (30 mg) for O₂-promoted methane photo-oxidation

| Reaction | Yield, | Selectivity, % | | | |
|---|---|---|---|---|---|
| time, h | μmol/g | CH₃OH | CH₃OOH | HCHO | HCOOH |
| 2 | 4.28 | 17.76 | 54.21 | 0 | 28.04 |
| 4 | 4.32 | 14.81 | 21.30 | 0 | 63.89 |
| 6 | 6.12 | 20.26 | 28.76 | 0 | 50.98 |
| 8 | 8.44 | 13.74 | 8.06 | 0 | 78.20 |
| 10 | 11.96 | 10.03 | 4.68 | 0 | 85.28 |
| 18 | 17.19 | 7.21 | 1.40 | 9.70 | 81.69 |
| 20 | 20.01 | 9.79 | 3.80 | 6.66 | 79.75 |

The stability of the GaN catalyst over multiple cycles of production was assessed. Fresh catalyst (30 mg of GaN powder) was used for the photocatalytic methane oxidation reactions with the following reaction conditions for each cycle: 1 mL of H₂O, 8 mL of O₂ (0.7 bar), 3 mL (0.3 bar) of CH₄, 25° C., 20 h (for each cycle), and irradiation with the 300 W Xe lamp. Upon completion of the first reaction cycle, the catalyst was separated from the reaction solution by a high-speed centrifuge and the catalyst was washed by distilled water 5 times in order to remove the surface-adsorbed reactants. Subsequently 1 mL of deionized water was added into the sample, which was then tested for another catalytic activity evaluation under the same reaction conditions, representing the second cycle of the catalyst. The remaining cycles were performed under the same conditions separated by the washing step. The process was repeated six times for a total of six cycles. Through the 6 cycles the same catalyst was therefore used for a total of 120 h of reaction time. The results of the first cycle are shown in FIG. 4C and the results of the 6 cycles are shown in FIG. 4D.

The switching of selectivity from formic acid to methanol was achieved by gradually decreasing the amount of O₂ in the GaN-catalyzed photo-conversion of methane in water. The experiment was performed with different oxygen levels: 0, 2, 4, 6, and 8 mL (i.e. 0, 0.175, 0.35, 0.525, and 0.7 bar respectively). The remaining reaction conditions were as follows: 30 mg of catalyst, 1 mL of H₂O, 120 μmol of CH₄ under 1 atm, 25° C., and irradiation by the 300 W Xe lamp. The results are presented in FIG. 4E and Table 7.

TABLE 7
The yields and selectivity of products over
20 h of GaN with different O₂ amounts

| O₂, | Yield, | Selectivity, % | | | | |
|---|---|---|---|---|---|---|
| bar | μmol/g | HCOOH | CH₃OH | CH₃OOH | HCHO | HOCH₂OOH |
| 0 | 17.96 | 9.35 | 90.65 | 0 | 0 | 0 |
| 0.175 | 14.80 | 60.00 | 25.41 | 14.59 | 0 | 0 |
| 0.35 | 13.02 | 70.05 | 19.35 | 7.37 | 3.23 | 0 |
| 0.525 | 17.83 | 82.10 | 6.28 | 0 | 5.23 | 6.39 |
| 0.7 | 20.01 | 79.75 | 9.79 | 3.80 | 6.66 | 0 |

In absence of O₂, under otherwise the same conditions, a continuous production of CH₃OH with 90.65% selectivity was achieved and maintained throughout the 20 h of reaction course (FIG. 4D and Table 8). The corresponding yield of total C1 oxygenates reaches up to 17.96 μmol g cat-1, which is comparable to the one under O₂. The GaN semiconductor thus enabled dual selective conversions of methane into formic acid and methanol, respectively, under aqueous media.

TABLE 8
The time-dependent reaction under GaN mass (30 mg)
for O₂-free (0 mL oxygen) methane photo-oxidation

| Reaction | Yield, | Selectivity, % | | | |
|---|---|---|---|---|---|
| time, h | μmol/g | CH₃OH | CH₃OOH | HCHO | HCOOH |
| 2 | 2.56 | 71.88 | 0 | 0 | 28.13 |
| 4 | 3.52 | 69.32 | 0 | 0 | 30.68 |
| 6 | 3.80 | 57.89 | 13.68 | 0 | 28.42 |
| 8 | 5.28 | 74.24 | 7.58 | 0 | 18.18 |
| 10 | 6.08 | 69.08 | 5.26 | 0 | 25.66 |
| 18 | 14.92 | 78.55 | 10.19 | 0 | 11.26 |
| 20 | 17.96 | 90.65 | 0 | 0 | 9.35 |

For comparison, the photocatalytic performances of ZnO, TiO₂ and g-C₃N₄ as other representative semiconductors used commonly for methane oxidations in the art were investigated under identical conditions of oxygen, catalyst mass, solvent volume, temperature and irradiation as follows: 30 mg catalyst, 1 mL of H₂O, 8 mL of O₂ (0.7 bar), 3 mL (0.3 bar) of CH₄, 25° C., and irradiation with the 300 W Xe lam. All three bare semiconductors showed much lower yields of oxygenates compared to GaN, among which the best performing catalyst is g-C₃N₄ with merely 5.08 μmol g⁻¹ cat, which is approximately one third of the value obtained for GaN (FIG. 4G and Table 9).

TABLE 9
The yields and selectivity of products over 20 h with GaN, ZnO,
g-C₃N₄, Ga₂O₃ and TiO₂ for selective O₂-promoted methane photo-oxidation

| | Yield, | Selectivity, % | | | | | |
|---|---|---|---|---|---|---|---|
| Samples | μmol/g | HCOOH | CH₃OH | CH₃OOH | HCHO | CH₃CH₂OH | CO₂ |
| GaN | 20.01 | 79.75 | 9.79 | 3.80 | 6.66 | 0 | n.d. |
| ZnO | 8.76 | 0 | 0 | 16.44 | 62.67 | 0 | 20.89 |
| g-C₃N₄ | 12.13 | 18.80 | 4.62 | 0 | 6.60 | 11.87 | 58.11 |
| Ga₂O₃ | 47.37 | trace | 1.01 | 1.84 | 0.78 | 0 | 96.37 |
| TiO₂ | n.d. | | | | | | |

Furthermore, the over-oxidation products of CO₂ from photo-conversion of methane with water and O₂ over g-C₃N₄ was clearly observed after 20 h reaction while being barely noticeable in the GaN-catalyzed system (FIG. 4G, FIG. 4H and Table 9). It is generally believed that because its oxidation potential is not enough to oxidize methane molecule, indirect activation of methane occurs on the surface of g-C₃N₄ semiconductor upon excitation with the assistant of water-generated oxidative radicals. In sharp contrast, UV-sensitive GaN was able to grant easy access to methyl radical via the direct methane activation due to its strong oxidizing ability. The results were also consistent with GaN's lower activation energy (24.49 KJ mol⁻¹) rather than that of g-C₃N₄ (53.16 KJ mol⁻¹) as calculated between 25° C.˜55° C. (FIG. 4I). These comparison experiments elucidated the advantage of GaN semiconductor in integrating direct and indirect activation of methane together, allowing photo-induced conversion of methane based on GaN with both high reactivity and product selectivity.

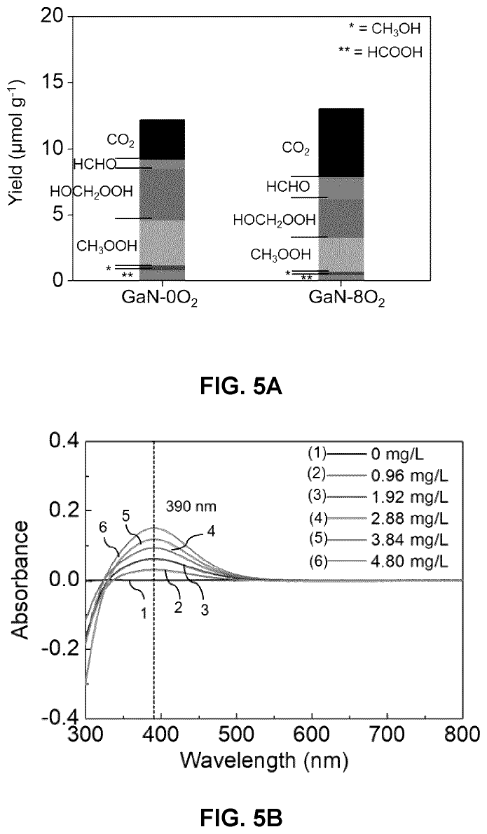

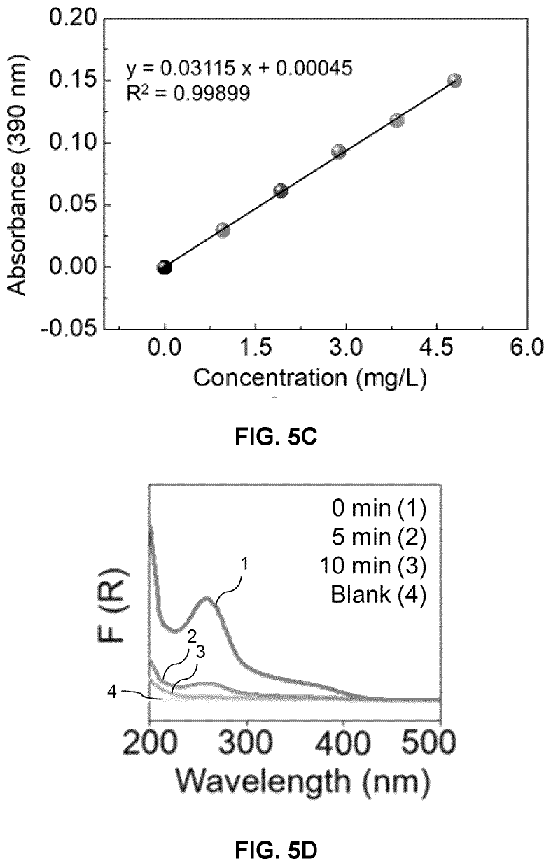

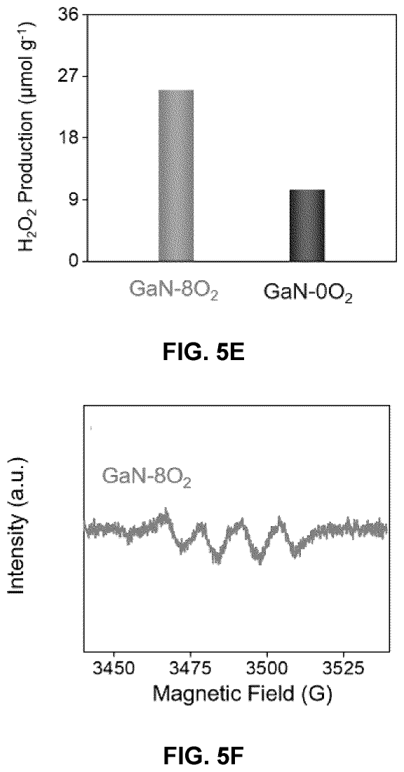

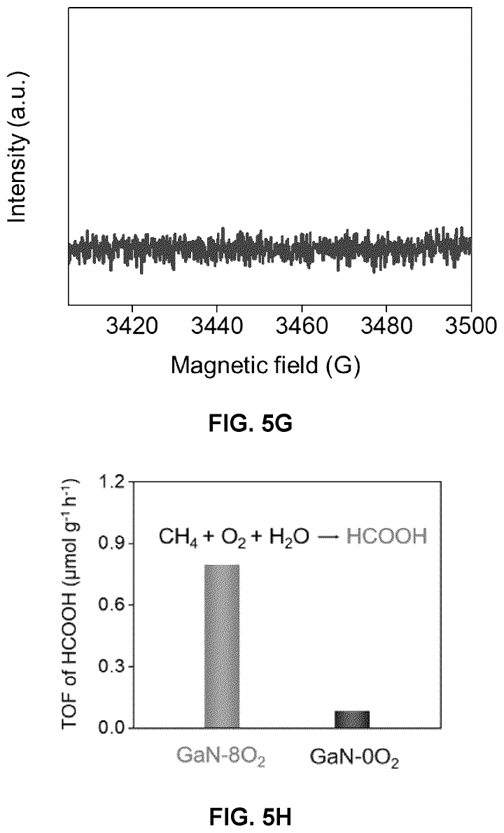

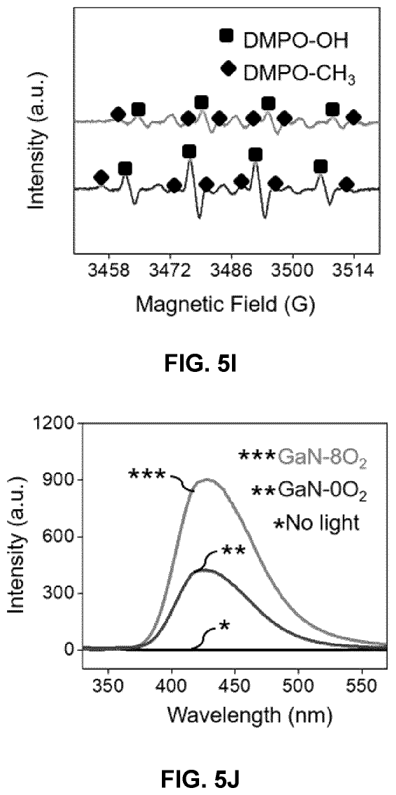

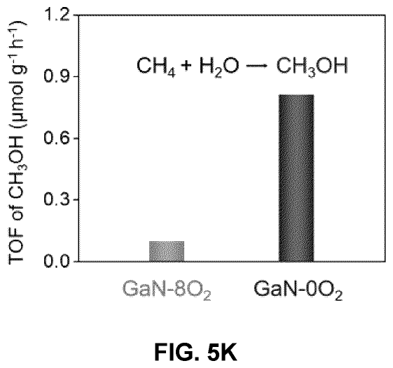

Utilizing additional H₂O₂ as oxidant to trigger and facilitate methane cleavage for accessing methanol and methyl hydroperoxide through methane-methyl hydroperoxide-methanol pathway has emerged in low-temperature methane thermal conversion. However, introducing additional H₂O₂ into the present photo-conversion system, regardless of O₂-promoted or O₂-free conditions, surprisingly did not work so efficiently as previously reported examples. A catalysis with the following reaction conditions was performed: 30 mg catalyst, 1 mL of H₂O (including 15 UL 30% H₂O₂), 8 or 0 mL of O₂ (0.7 bar or 0 bar), 3 mL (0.3 bar) of CH₄, 25° C., and irradiation with the 300 W Xe lamp. The production of CH₃OOH and HOCH₂OOH reaction intermediate indicated that the selective methane photo-conversions undergo a hydroperoxyl event (FIG. 5A and Table 10). Meanwhile, a notable drop in total yield of total liquid oxygenates and selectivity to either desired formic acid or methanol further established the irreplaceable role of in situ generated H₂O₂ on the GaN catalyst surface, rather than external H₂O₂, in boosting the selective generation of methanol and formic acid (FIG. 5A and Table 10).

TABLE 10
The yields and selectivity of products over 20 h with GaN for selective
O₂-promoted and O₂-free methane photooxidation with adding H₂O₂

| | | Yield, | Selectivity, % | | | | | |
|---|---|---|---|---|---|---|---|---|
| Sample | O₂, bar | μmol/g | HCOOH | CH₃OH | CH₃OOH | HCHO | HOCH₂OOH | CO₂ |
| GaN | 0 | 14.38 | 5.84 | 2.23 | 23.93 | 4.87 | 27.13 | 36.00 |
| GaN | 0.7 | 10.53 | 4.56 | 1.90 | 24.68 | 12.34 | 27.91 | 28.61 |

The amount of H₂O₂ was determined by a potassium titanium oxalate spectrophotometric method. Briefly, 1 mL filtrate liquid products from the suspension after 20 h irradiation was added into chromogenic reagent containing 1 mL of 0.12 mol L⁻¹ potassium titanium oxalate solution and 1 mL of 0.055 mol L⁻¹ sulfuric acid. The absorbance at 390 nm was detected on the Uv-vis spectrophotometer.

A moderate H₂O₂ yield of 24.99 μmol g cat⁻¹ was observed, catalyzed by GaN via two-electron O₂ reduction under O₂-promoted condition, which was 2-fold that of O₂-free methane photo-oxidation (10.48 μmol g cat⁻¹). The O₂-promoted relatively higher generation of H₂O₂ is believed to be beneficial for the subsequent stage of generating active ·OOH radical (FIG. 5B-5C). Indeed, comparing to without O₂, a more pronounced ·OOH radical signal was identified using 5, 5-dimethyl-1-pyrroline N-oxide (DMPO) as spin trap after a short-time UV-illumination with O₂ as co-oxidant (FIG. 5D and FIG. 5E). The electron paramagnetic resonance (EPR) spectroscopy measurement for DMPO was obtained using a Bruker™ Elexsys E580 X-band EPR Spectrometer. It was found that O₂-promoted in situ generation of ·OOH radical, which was enriched on the surface of GaN, and had a positive influence on pushing the catalytic process towards the synthesis of deep oxidation product (formic acid) while avoiding over-oxidation to CO₂ (FIG. 5F).

For the hydroxyl radical (·OH) detection, the formation of ·OH was monitored with terephthalic acid (98% purity, Sigma-Aldrich) as a probe, which could readily capture the radical to produce fluorescent 2-hydroxyterephthalic acid (Y. Zhou, L. Zhang, W. Wang, Direct functionalization of methane into ethanol over copper modified polymeric carbon nitride via photocatalysis. Nat. Commun. 10, 1-8 (2019)). The following procedure was carried out. 30 mg catalyst was dispersed in 1 mL of 0.5 mmol L⁻¹ terephthalic acid dissolved into 2 mmol L⁻¹ sodium hydroxide and kept stirring, then the reactor was frozen by liquid nitrogen (N_{2(liquid)} at 196° C. for 10 s), followed by the introduction of 0.3 bar CH₄ gas (3 mL) and corresponding amount of O₂ gas (0 or 0.7 bar, 0 or 8 mL) with syringes under room temperature. Fluorescence spectra of 2-hydroxyterephthalic acid after 1 h illumination was measured by spectrophotometer excited at 315 nm.

For the superoxide radical (·OOH) detection, nitrotetrazolium blue chloride (NBT) was used as the probe molecule to detect ·OOH radicals (L. Luo, Z. Gong, Y. Xu, J. Ma, H. Liu, J. Xing, J. Tang, Binary Au—Cu Reaction Sites Decorated ZnO for Selective Methane Oxidation to C1 Oxygenates with Nearly 100% Selectivity at Room Temperature. J. Am. Chem. Soc. 144, 740-750 (2022)). Typically, 30 mg GaN powder was mixed with 1 mL NBT solution (0.04 mM) and stirred in dark for 30 min. After irradiation for a specific length of time (0 min, 5 min, 10 min), 1 mL of liquid product was mixed with 2.0 ml of water. The mixed solution was measured by UV-Vis adsorption spectroscopy.

In addition, further radical spin-trapping investigations found the concurrent existence of ·CH₃ and ·OH in the photocatalytic methane oxidation upon the irradiation of UV light, regardless of with or without O₂ (FIG. 5G). Quantification analysis showed that more GaN-semiconductor photo-induced ·OH was generated with the increase of O₂ concentration due to the decomposition of more H₂O₂ (FIGS. 5A and 5H). However, the yield of primary oxygenates did not increase when increasing O₂, thus suggesting that ·CH₃ radical mainly results from the direct activation of methane triggered by photo-generated GaN hole (FIG. 3C and Table 3). Considering the rapid coupling of ·CH₃ with moderate ·OH, selective production of methanol occurred in the case of O₂-free methane photo-oxidation with a TOF of 0.814 μmol g cat⁻¹ h⁻¹ for methanol, which is 8 times higher than that in the O₂-involved case (0.098 μmol g cat⁻¹ h⁻¹) (FIG. 5l). Therefore, it can be concluded that the concentration of reactive oxygen radical can be varied readily by varying the concentration of O₂, allowing to control the selective generation of oxygenated products.

Based on the experimental results, a tentative mechanism of the photocatalytic oxidation of CH₄ over commercial GaN powder was proposed (Table 11). The introduction of O₂ could lead to the formation of reactive ·OOH radicals at the bottom of the conductive band (CB) of GaN powder (equation (1)). Meanwhile, CH₄ could be activated by GaN to form ·CH₃ radicals. Subsequently, the combination of ·CH₃ radicals and ·OOH radicals resulted in the formation of CH₃OOH (equation (2)), which could undergo a two-electron reduction process to generate CH₃OH (equation (3)). Then, the generated ·OH from H₂O₂ and H₂O would further combine with CH₃OH to form deep oxidation products HCOOH. In another case, without external O₂, H₂O firstly went through an oxidation process to generate H₂O₂ by the holes on the valence band of GaN, and could be further reduced to ·OH radicals in situ by the electrons excited to the conduction band after the illumination. At the same time, the formed ·OH radicals combined with the ·CH₃ radicals to form CH₃OH, in which the generation rate of ·OH and ·CH₃ radicals has limited the extent of oxidation process.

TABLE 11
Reaction equations of the proposed reaction mechanism for
selective O₂-promoted and O₂-free methane photo-oxidation

| Equation number | Reaction equations |
|---|---|
| 1 | O₂ + H⁺ + e⁻ → •OOH |
| 2 | •OOH + •CH₃ → CH₃OOH |
| 3 | CH₃OOH + 2H⁺ + 2e⁻ → CH₃OH + H₂O |
| 4 | H₂O₂ + H⁺ + 2e⁻ → •OH + H₂O |
| 5 | •OH + •CH₃ → CH₃OH |

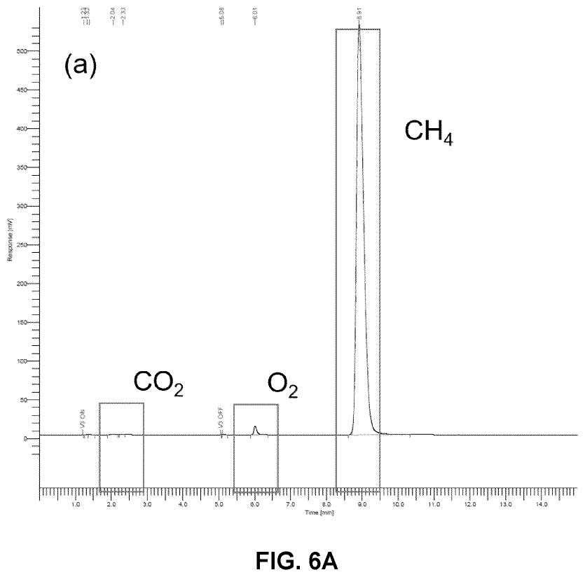

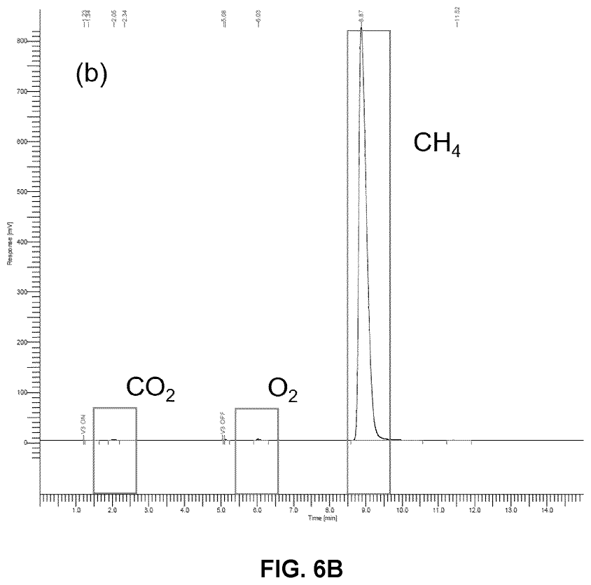

Additional analysis to quantify whether any CO₂ or CO was produced as by-products was performed by gas chromatography with flame-ionization detection (GC-FID) since GC-FID is more sensitive than GC-TCD. In brief, the protocol was as follows: 10 mL of gas products, obtained under the reaction conditions (100 mg GaN, 10 mL H₂O, 0.3 bar CH₄, 0.7 bar O₂, 2 h reaction length under a 300 W Xe lamp), was analyzed by manual injection using gas chromatograph (GC, Clarus 590) equipped with methanizer and flame ionization detector. As a negative control, the GC-FID spectra of a gas sample obtained without catalyst and light was performed (FIG. 6A). In comparison, the sample obtained under the reaction conditions mentioned above showed no or negligible CO and CO₂ in the products of the reaction (FIG. 6B).

For the apparent quantum efficiency (AQE) measurement, because the Xe lamp used in the standard procedure has a wide wavelength spectrum, another single wavelength 370 nm kessil lamp was used to conduct the experiment to measure the AQE of GaN under the reaction condition: 100 mg GaN, 10 mL H₂O, 0.3 bar CH₄/0.7 bar O₂ and 0.3 bar CH₄/0 bar O₂, for 48 h. The irradiation area was controlled to be Π* (2.5)² cm², and the light intensity was measured to be 12.98 mW·cm⁻² by a Model 843-R power meter (Newport Corporation) equipped with an 818-ST2-UV power detector. The AQE was calculated by the following equations:

$$AQE = \frac{{N}_{e}}{{N}_{p}} * 100 \% = \frac{1 {0}^{9} ( {N}_{A} * v * K ) * ( h * c )}{( 1 * A * λ )}$$

Ne is the number of reaction electrons, Np is the number of incident photons, NA is Avogadro's constant (6.02×10²³ mol⁻¹), v is reaction rate (mol s⁻¹), K is the charge transfer numbers, h is the Planck constant (6.62×10⁻³⁴ J·s), c is the speed of light (3.0×10⁸ m·s⁻¹), I is the intensity of the irradiation (W·m⁻²), A is the irradiation area (m²), and λ is the wavelength of the monochromatic light (nm).

The AQE of commercial GaN for the synthesis of HCOOH/CH₃OH in O₂/O₂-free condition turns was found to be 0.009% and 0.013%, respectively.

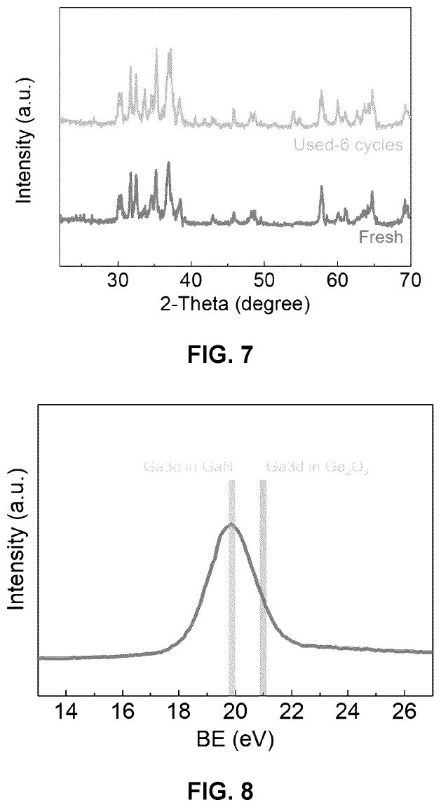

As previously demonstrated in FIG. 4D, the catalyst can be used for multiple cycles. The structural stability of the GaN was investigated by XRD. A GaN after 6 reaction cycles was compared to a fresh GaN having performed zero cycles (FIG. 7). As can be seen from FIG. 7, the GaN retains its structure as the XRD spectra are almost identical before any reaction and after 6 cycles (FIG. 7).

Finally, the surface of GaN was investigated by XPS. The band structure of GaN sample is almost consistent with the theoretical value. More importantly, XPS Ga 3d peak of GaN in the high-resolution spectrum does not show an obvious shift to high binding energy (FIG. 8) which indicates that the main component on the sample surface is still GaN rather than a Ga₂O₃/GaN interface. These results demonstrate that Ga₂O₃/GaN heterojunctions do not significantly contribute to the catalytical performance.

While the disclosure has been described with particular reference to the illustrated embodiment, it will be understood that numerous modifications thereto will appear to those skilled in the art. Accordingly, the above description and accompanying drawings should be taken as illustrative and not limitative in any sense.

1. A method of producing C1 oxygenated products comprising:
providing an aqueous phase and GaN in a reactor having a closed environment;
introducing from 0.1 to 5 bar of methane and from 0 to 10 bar of oxygen in the reactor; and
irradiating the reactor with ultra-violet light until C1 oxygenated products are obtained.

2. The method of claim 1, wherein the C1 oxygenated products are methanol and/or formic acid.

3. The method of claim 1, wherein the closed environment of the reactor is evacuated before the step of introducing the methane and the oxygen.

4. The method of claim 3, wherein the closed environment is evacuated to a pressure of less than 0.001 bar.

5. The method of claim 1, wherein the closed environment of the reactor is purged with methane and optionally oxygen.

6. The method of claim 1, wherein the temperature during the step of irradiating the reactor is from 20 to 55° C.

7. The method of claim 1, wherein the ultra-violet light has a wavelength of from 200 to 410 nm.

8. The method of claim 1, wherein the aqueous phase is an aqueous suspension comprising the GaN.

9. The method of claim 8, wherein the aqueous suspension comprises from 5 to 30 g/L of the GaN.

10. The method of claim 1, wherein the GaN is supported on a zeolite or a silica gel and the reactor is a flow reactor.

11. The method of claim 3, further comprising freezing the reactor before the reactor is evacuated or purged.

12. The method of claim 1, further comprising separating the C1 oxygenated products.

13. The method of claim 1, wherein the C1 oxygenated products comprise at least 85% by weight of methanol.

14. The method of claim 13, wherein less than 0.1 bar of oxygen is introduced in the reactor.

15. The method of claim 13, wherein no oxygen is introduced in the reactor.

16. The method of claim 1, further comprising separating methanol.

17. The method of claim 1, wherein the C1 oxygenated products comprise at least 70% by weight forming acid.

18. The method of claim 17, wherein at least 0.35 bar of oxygen is introduced in the reactor.

19. The method of claim 1, further comprising separating formic acid.

20. The method of claim 17, wherein H₂O₂ is produced in situ at the surface of GaN.
